(19) United States
(12) Patent Application Publication
(10) Pub. No.: US 2018/0148683 A1
(43) Pub. Date: May 31, 2018

(54) INDUCTION OF GENE EXPRESSION USING A HIGH CONCENTRATION SUGAR MIXTURE

(71) Applicant: DANISCO US INC., Palo Alto, CA (US)

(72) Inventors: Timothy C. DODGE, Sunnyvale, CA (US); Ilkka Ilari KRUUS, Wilmington, CA (US); Colin MITCHINSON, Half Moon Bay, CA (US); Timo Tapio VILJAVA, Kantvik (FI)

(73) Assignee: DANISCO US INC., Palo Alto, CA (US)

(21) Appl. No.: 15/577,505

(22) PCT Filed: Jul. 7, 2016
(86) PCT No.: PCT/US2016/041292

§ 371 Date: Nov. 28, 2017

Related U.S. Application Data
(60) Provisional application No. 62/189,462, filed on Jul. 7, 2015.

(51) Int. Cl. C12N 1/38; C12P 21/00

(52) U.S. Cl. CPC C12N 1/38; C12P 21/00

(57) ABSTRACT

Described herein is a composition useful for inducing expression of genes whose expression is under control of an inducible promoter sequence and methods for the compositions preparation and use.

### CROSS-REFERENCE TO RELATED APPLICATIONS

The instant application claims priority to U.S. Provisional Patent Application Ser. No. 62/189,462, filed Jul. 7, 2015, which is hereby incorporated by reference in its entirety.

### FIELD OF THE INVENTION

This invention relates to methods for improved production of proteins from a cell culture. The invention also pertains to culture components and conditions that increase the amount of protein produced from genes under the control of inducible gene promoter. The improved methods can be used for the production of proteins encoded by naturally occurring cellulase genes as well as from various heterologous constructs.

### BACKGROUND OF THE INVENTION

Filamentous fungi and cellulolytic bacteria produce extracellular cellulase enzymes that confer on the organisms the ability to hydrolyze the β-(1,4)-linked glycosidic bonds of cellulose to produce glucose. These enzymes provide the organisms with the ability to use cellulose, the most abundant plant polysaccharide, for growth.

The filamentous fungus Trichoderma reesei (T. reesei; an anamorph of the fungus Hypocrea jecorina) is an efficient producer of cellulase enzymes. As such, T. reesei has been exploited for its ability to produce these enzymes, which are valuable in the production of such commodities as fuel ethanol, clothing, detergents, fibers and other products.

Expression and production of the main cellulase genes in Trichoderma, cbh1, cbh2, egl1, and egl2, is dependent on the carbon source available for growth. The cellulase genes are tightly repressed by glucose and are induced several thousand folds by cellulose or disaccharide, e.g., sophorose, lactose or gentiobiose. Published U.S. Pat. No. 7,713,725 disclosed a whole cellulase preparation added to a concentrated glucose solution with the sufficient incubation, the resulting complex mixture is able to induce cellulase production without further purification.

Sucrose, commonly named table sugar or sugar, is made from sugar cane and sugar beet. Sucrose sources including sucrose molasses, an important residue of the sugar industry, are abundantly available in some parts of the world, e.g., Brazil, India, European Union, China, Thailand, and United States. In such parts where sucrose sources are abundant and inexpensive, the cost of glucose can be deemed relatively very expensive especially in situation where large amounts or volumes of such sugar source is required to produce an inducer for use in large, commercial scale operations. Therefore, a need exists for a convenient, soluble substrate composition comprising sucrose that also provides an inexpensive method of cellulase induction in filamentous fungi, e.g., Trichoderma reesei, for use in commercial operations located in areas where sucrose is the most abundant and cost-effective sugar source.

### BRIEF SUMMARY OF THE INVENTION

This invention relates to methods for improved production of proteins from a cell culture. The invention also pertains to culture components and conditions that increase the amount of protein produced from genes under the control of inducible gene promoter. The improved methods can be used for the production of proteins encoded by naturally occurring cellulase genes as well as from various heterologous constructs.

In a first aspect, the present disclosure provides a method of producing an inducing feed composition, said method comprising the steps of: (a) generating a first mixture comprising a first solution comprising sucrose and at least one inverting enzyme; and (b) incubating the first mixture at a temperature for a sufficient time to produce an inverted mixture; and (c) generating a second mixture comprising the inverted mixture produced from (b) and at least one reverting enzyme; and (d) incubating the second mixture at a temperature for a sufficient time to produce the inducing feed composition.

In certain embodiments, the first solution comprises a Sugarcane Juice Syrup (SJS).

In certain other embodiments, the first solution comprises a Very High Purity Sucrose (VHP).

In some embodiments, the first solution comprises a Molasses (Mol).

In certain further embodiments, the inverting enzyme of this aspect is an invertase.

In any of the above embodiments, the first mixture is incubated at a temperature within the range of from about 30° C. to about 100° C. for a period of between 1 hour and 60 hours. For example, the first mixture may be incubated at a temperature from about 50° C. to about 80° C. for between 2 hours and 30 hours.

In any of the above embodiments of the method of the first aspect, the inverting enzyme may be a whole cellulase composition comprising a beta-glucosidase. For example, the reverting enzyme may be a beta-glucosidase-enriched cellulase composition.

In yet further embodiments, the second mixture of the method of this aspect is incubated at a temperature falling within the range of from about 30° C. to about 100° C. for a period of between 2 hours and 72 hours. For example, the second mixture may be incubated at a temperature within the range of from about 30° C. to about 90° C. (such as, e.g., about 40° C. to about 90° C., about 40° C. to about 80° C., about 50° C. to about 80° C., etc), for a period between 2 hours and 65 hours (such as, e.g., between 2 hours and 60 hours, between 5 hours and 55 hours, between 10 hours and 50 hours, etc.).

In a second aspect, the present disclosure provides an inducing feed composition, produced by applying the method of any of the embodiments of the first aspect above.

In some embodiments, the inducing feed composition comprises a mixture of sugars.

In certain embodiments, the inducing feed composition further comprises sophorose.

In certain further embodiments, the inducing feed composition may comprise gentiobiose.

In a third aspect, the present disclosure provides a method for producing a protein of interest from a cell culture comprising the steps of: first, producing an inducing feed composition following the steps of (i) incubating a solution comprising from about 50% to about 70% sucrose and at least one inverting enzyme, generating a first mixture; and (ii), incubating the first mixture at a suitable temperature for a sufficient time period to produce an inverted mixture; (iii) generating a second mixture by combining the inverting mixture produced from (ii), and at least one reverting enzyme; and (iv) incubating the second mixture at a suitable temperature for a sufficient period of time to produce an inducing feed composition; and second, contacting the cell culture, which comprises cells comprising a nucleotide sequence encoding a protein is interest operatively linked to an inducible promoter, with the inducing feed composition produced by the steps above, in an amount effective to induce expression the protein of interest by the cell culture.

In some embodiments of the method of this aspect, the first mixture is incubated at a temperature ranging from about 30° C. to about 100° C. for a period of between 1 hour and 60 hours. For example, the first mixture may be incubated at a temperature falling in the range of from about 30° C. to about 90° C. (such as, e.g., about 30° C. to about 80° C., about 40° C. to about 90° C., about 40° C. to about 80° C., about 50° C. to about 80° C., etc.), for a period of time between 2 hours and 60 hours (such as, e.g., between 2 hours and 55 hours, between 5 hours and 60 hours, between 10 hours and 50 hours, etc.).

In some embodiment of the method of this aspect, the second mixture is incubated at a temperature ranging from about 30° C. to about 100° C., for a period of between 2 hours and 72 hours. For example, the second mixture may be incubated at a temperature falling within the range of ranging from about 30° C. to about 90° C. (such as, e.g., about 30° C. to about 80° C., about 40° C. to about 90° C., about 40° C. to about 80° C., about 40° C. to about 70° C., about 50° C. to about 80° C., etc.), for a period of between 2 hours and 70 hours (such as, e.g., between 5 hours and 65 hours, between 10 hours and 60 hours, between 12 hours and 55 hours, etc.).

In certain embodiments of the method of this aspect, the protein of interest is an endogenous protein.

In certain further embodiments, the protein of interest is a heterologous protein.

In some embodiments, the protein of interest is selected from the group consisting of enzymes, hormones, growth factors, cytokines, and antibodies. For example, the protein of interest may be one of hemicellulases, peroxidases, proteases, cellulases, xylanases, lipases, phospholipases, esterases, cutinases, pectinases, keratinases, reductases, oxidases, phenol oxidases, lipoxygenases, ligninases, pullulanases, tannases, pentosanases, manannase, beta-glucanases, arabinosidases, hyaluronidase, chondroitinase, laccase, amylases, glucoamylases, and mixtures thereof.

In some embodiments, the inducible promoter is a sophorose-inducible promoter.

In some embodiments, the inducible promoter is a gentiobiose-inducible promoter.

In some embodiments, the promoter is a cellulase gene promoter. For example, the promoter may be a cbh 1 promoter from Trichoderma reesei.

In some embodiments, the cell of the cell culture is a filamentous fungal cell. For example, the filamentous fungal cell is one that is selected from the group of Trichoderma, Humicola, Fusarium, Aspergillus, Neurospora, Penicillium, Cephalosporium, Achlya, Podospora, Endothia, Mucor, Cochliobolus, Myceliophthora, or Pyricularia. The fungus may be one of a Trichoderma spp., a Myceliophthora spp., a Penicillium spp. or an Aspergillus spp.

In some embodiments, the cell of the cell culture is a bacterial cell. For example, the bacterial cell may be one derived from a bacterium selected from Streptomyces, Thermomonospora, Bacillus, or Cellulomonas.

### BRIEF DESCRIPTION OF THE DRAWINGS

FIG. 1 shows the SDS-PAGE analysis of the protein products of each of the fermentations. The protein samples were diluted to 5 mg/mL based on the total protein measured by the modified Biuret method. The samples were prepared according to standard NuPAGE® protocols (Life Technologies) with LDS sample buffer, and run on a NuPAGE® 4-12% Bis-Tris gel with MOPS buffer.

### DETAILED DESCRIPTION

### I. Overview

This invention relates to methods for improved production of proteins from a cell culture. The invention also pertains to culture components and conditions that increase the amount of protein produced from genes under the control of inducible gene promoter. The improved methods can be used for the production of proteins encoded by naturally occurring cellulase genes as well as from various heterologous constructs.

### II. Abbreviations and Acronyms

The following abbreviations/acronyms have the following meanings unless otherwise specified:

cDNA complementary DNA

DNA deoxyribonucleic acid

kDa kiloDalton

MW molecular weight

RNA ribonucleic acid

SDS-PAGE sodium dodecyl sulfate polyacrylamide gel electrophoresis

sp. species

Tm melting temperature

w/v weight/volume

w/w weight/weight

° C. degrees Centigrade

H₂O water

g or gm grams

μg micrograms

mg milligrams

kg kilograms

μL and μl microliters

mL and ml milliliters

mm millimeters

μm micrometer

M molar

mM millimolar

μM micromolar

U units

sec seconds

min(s) minute/minutes

hr(s) hour/hours

Tris-HCl tris(hydroxymethyl)aminomethane hydrochloride

MOPS 3-(N-morpholino)propanesulfonic acid

### III. Definitions

Prior to describing the present compositions and methods, the following terms and phrases are defined. Terms not defined should be accorded their ordinary meaning as used in the art.

Where a range of values is provided, it is understood that each intervening value, to the tenth of the unit of the lower limit unless the context clearly dictates otherwise, between the upper and lower limit of that range and any other stated or intervening value in that stated range, is encompassed within the present compositions and methods. The upper and lower limits of these smaller ranges may independently be included in the smaller ranges and are also encompassed within the present compositions and methods, subject to any specifically excluded limit in the stated range. Where the stated range includes one or both of the limits, ranges excluding either or both of those included limits are also included in the present compositions and methods.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number. For example, in connection with a numerical value, the term “about” refers to a range of −10% to +10% of the numerical value, unless the term is otherwise specifically defined in context. In another example, the phrase a “pH value of about 6” refers to pH values of from 5.4 to 6.6, unless the pH value is specifically defined otherwise.

The headings provided herein are not limitations of the various aspects or embodiments of the present compositions and methods which can be had by reference to the specification as a whole. Accordingly, the terms defined immediately below are more fully defined by reference to the specification as a whole.

The present document is organized into a number of sections for ease of reading; however, the reader will appreciate that statements made in one section may apply to other sections. In this manner, the headings used for different sections of the disclosure should not be construed as limiting.

In accordance with this detailed description, the following abbreviations and definitions apply. Note that the singular forms “a,” “an,” and “the” include plural referents unless the context clearly dictates otherwise. Thus, for example, reference to “an enzyme” includes a plurality of such enzymes, and reference to “the dosage” includes reference to one or more dosages and equivalents thereof known to those skilled in the art, and so forth.

It is further noted that the claims may be drafted to exclude any optional element. As such, this statement is intended to serve as antecedent basis for use of such exclusive terminology as “solely,” “only,” and the like in connection with the recitation of claim elements, or use of a “negative” limitation.

It is further noted that the term “consisting essentially of,” as used herein refers to a composition wherein the component(s) after the term is in the presence of other known component(s) in a total amount that is less than 30% by weight of the total composition and do not contribute to or interferes with the actions or activities of the component(s).

It is further noted that the term “comprising,” as used herein, means including, but not limited to, the component(s) after the term “comprising.” The component(s) after the term “comprising” are required or mandatory, but the composition comprising the component(s) may further include other non-mandatory or optional component(s).

It is also noted that the term “consisting of” as used herein, means including, and limited to, the component(s) after the term “consisting of.” The component(s) after the term “consisting of” are therefore required or mandatory, and no other component(s) are present in the composition.

As will be apparent to those of skill in the art upon reading this disclosure, each of the individual embodiments described and illustrated herein has discrete components and features which may be readily separated from or combined with the features of any of the other several embodiments without departing from the scope or spirit of the present compositions and methods described herein. Any recited method can be carried out in the order of events recited or in any other order which is logically possible.

As used herein, the term “antibody” refers to a polypeptide comprising a framework region from an immunoglobulin gene or fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. Typically, the antigen-binding region of an antibody or its functional equivalent will be most critical in specificity and affinity of binding. See Paul, Fundamental Immunology.

An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (VL) and variable heavy chain (VH) refer to these light and heavy chains respectively.

As used herein, the term “cellulase,” “cellulolytic enzymes” or “cellulase enzymes” means bacterial, or fungal exoglucanases or exocellobiohydrolases, and/or endoglucanases, and/or β-glucosidases. These three different types of cellulase enzymes act synergistically to convert cellulose and its derivatives to glucose.

Many microbes make enzymes that hydrolyze cellulose, including the wood rotting fungus Trichoderma, the compost bacteria Thermomonospora (now Thermobifida), Bacillus, and Cellulomonas; Streptomyces; and the fungi Humicola, Aspergillus and Fusarium. The enzymes made by these microbes are mixtures of proteins with three types of actions useful in the conversion of cellulose to glucose: endoglucanases (EG), cellobiohydrolases (CBH), and beta-glucosidase (BG).

As used herein, the phrases “whole cellulase preparation” and “whole cellulase composition” are used interchangeably and refer to both naturally occurring and non-naturally occurring compositions. A “naturally occurring” composition is one produced by a naturally occurring source and which comprises one or more cellobiohydrolase-type, one or more endoglucanase-type, and one or more β-glucosidase components wherein each of these components is found at the ratio produced by the source. A naturally occurring composition is one that is produced by an organism unmodified with respect to the cellulolytic enzymes such that the ratio of the component enzymes is unaltered from that produced by the native organism.

As used herein, a “non-naturally occurring” composition encompasses those compositions produced by: (1) combining component cellulolytic enzymes either in a naturally occurring ratio or non-naturally occurring, i.e., altered, ratio; or (2) modifying an organism to overexpress or underexpress one or more cellulolytic enzyme; or (3) modifying an organism such that at least one cellulolytic enzyme is deleted.

The whole cellulase mixtures useful in the present invention may have one or more of the various EGs and/or CBHs deleted. For example, EG1 may be deleted alone or in combination with other EGs and/or CBHs. BGs may be over-expressed relative to the native levels. Heterologous expression of BGs is also contemplated herein.

As used herein, the term “carbon limitation” is a state wherein a microorganism has just enough carbon to produce a desired protein product, but not enough carbon to completely satisfy the organism's requirement, e.g., sustain growth. Therefore, the maximal amount of carbon goes toward protein production.

As used herein, the terms “promoter” and “cellulase promoter” refer to a nucleic acid sequence that functions to direct transcription of a downstream gene and are used interchangeably herein. The promoter will generally be appropriate to the host cell in which the target gene is being expressed. The promoter together with other transcriptional and translational regulatory nucleic acid sequences (also termed “control sequences”) is necessary to express a given gene. In general, the transcriptional and translational regulatory sequences include, but are not limited to, promoter sequences, ribosomal binding sites, transcriptional start and stop sequences, translational start and stop sequences, and enhancer or activator sequences. In one aspect the promoter is an inducible promoter. In another aspect the promoter is inducible by an inducer selected from the group consisting of gentiobiose, cellulose and sophorose. In one aspect the promoter is the T. reesei cbh1 promoter which is deposited in GenBank under Accession Number D86235. In another aspect the promoter is a cbh II or xylanase promoter from T. reesei.

As used herein, a “promotor sequence” is a DNA sequence which is recognized by the particular filamentous fungus for expression purposes. A “promoter” is defined as an array of nucleic acid control sequences that direct transcription of a nucleic acid. As used herein, a promoter includes necessary nucleic acid sequences near the start site of transcription, such as, in the case of a polymerase II type promoter, a TATA element. A “constitutive” promoter is a promoter that is active under most environmental and developmental conditions. An “inducible” promoter is a promoter that is active under environmental or developmental regulation. An example of an inducible promoter useful in the present invention is the T. reesei cbh 1 promoter. The term “operably linked” refers to a functional linkage between a nucleic acid expression control sequence (such as a promoter, or array of transcription factor binding sites) and a second nucleic acid sequence, wherein the expression control sequence directs transcription of the nucleic acid corresponding to the second sequence.

Examples include the promoter from the A. awamori or A. niger glucoamylase genes (Nunberg, J. H. et al. (1984) Mol. Cell. Biol. 4, 2306-2315; Boel, E. et al. (1984) EMBO J. 3, 1581-1585), the Mucor miehei carboxyl protease gene herein, the Trichoderma reesei cellobiohydrolase I gene (Shoemaker, S. P. et al. (1984) European Patent Application No. EPO0137280A1), the A. nidulans trpC gene (Yelton, M. et al. (1984) Proc. Natl. Acad. Sci. USA 81, 1470-1474; Mullaney, E. J. et al. (1985) Mol. Gen. Genet. 199, 37-45) the A. nidulans alcA gene (Lockington, R. A. et al. (1986) Gene 33, 137-149), the A. nidulans tpiA gene (McKnight, G. L. et al. (1986) Cell 46, 143-147), the A. nidulans amdS gene (Hynes, M. J. et al. (1983) Mol. Cell Biol. 3, 1430-1439), the T. reesei xln1 gene, the T. reesei cbh2 gene, the T. reesei eg1 gene, the T. reesei egg gene, the T. reesei eg3 gene, and higher eukaryotic promoters such as the SV40 early promoter (Barclay, S. L. and E. Meller (1983) Molecular and Cellular Biology 3, 2117-2130).

A nucleic acid is “operably linked” when it is placed into a functional relationship with another nucleic acid sequence. For example, DNA encoding a secretory leader, i.e., a signal peptide, is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

As used herein, the term “gene” means the segment of DNA involved in producing a polypeptide chain, that may or may not include regions preceding and following the coding region, e.g. 5′ untranslated (5′ UTR) or “leader” sequences and 3′ UTR or “trailer” sequences, as well as intervening sequences (introns) between individual coding segments (exons).

The gene may encode therapeutically significant proteins or peptides, such as growth factors, cytokines, ligands, receptors and inhibitors, as well as vaccines and antibodies. The gene may encode commercially important industrial proteins or peptides, such as enzymes, e.g., proteases, mannanases, xylanases, amylases, glucoamylases, cellulases, oxidases and lipases. The gene of interest may be a naturally occurring gene, a mutated gene or a synthetic gene.

As used herein, the term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all.

As used herein, the term “secretory signal sequence” denotes a DNA sequence that encodes a polypeptide (a “secretory peptide”) that, as a component of a larger polypeptide, directs the larger polypeptide through a secretory pathway of a cell in which it is synthesized. The larger peptide is commonly cleaved to remove the secretory peptide during transit through the secretory pathway.

As used herein, the term “induction” refers to the increased transcription of a gene resulting in the synthesis of a protein of interest in a cell or organism at a markedly increased rate in response to the presence of an “inducer”. To measure the induction of a protein of interest, cells treated with a potential inducer are compared to control samples without the inducer. Control samples (untreated with inducers) are assigned a relative protein activity value of 100%. Induction of a polypeptide is achieved when the activity value relative to the control (untreated with inducers) is greater than 100%, greater than 110%, more preferably 150%, more preferably 200-500% (i.e., two to five fold higher relative to the control), or more preferably 1000-3000% higher.

As used herein, the term “filamentous fungi” of the present invention are eukaryotic microorganisms and include all filamentous forms of the subdivision Eumycotina (see Alexopoulos, C. J. (1962), Introductory Mycology, New York: Wiley). These fungi are characterized by a vegetative mycelium with a cell wall composed of chitin, cellulose, and other complex polysaccharides. The filamentous fungi of the present invention are morphologically, physiologically, and genetically distinct from yeasts. Vegetative growth by filamentous fungi is by hyphal elongation and carbon catabolism is obligately aerobic. In contrast, vegetative growth by yeasts such as S. cerevisiae is by budding of a unicellular thallus, and carbon catabolism may be fermentative. S. cerevisiae has a prominent, very stable diploid phase, whereas diploids exist only briefly prior to meiosis in filamentous fungi, e.g., Aspergillus and Neurospora. S. cervisiae has 17 chromosomes as opposed to 8 and 7 for A. nidulans and N. crassa respectively. Recent illustrations of differences between S. cerevisiae and filamentous fungi include the inability of S. cerevisiae to process Aspergillus and Trichoderma introns and the inability to recognize many transcriptional regulators of filamentous fungi (Innis, M. A. et al. (1985) Science, 228, 21-26).

As used herein, the term “glucosidases” refers to any enzyme whose end product is glucose.

As used herein, the term “heterologous” when used with reference to portions of a nucleic acid indicates that the nucleic acid comprises two or more subsequences that are not normally found in the same relationship to each other in nature. For instance, the nucleic acid is typically recombinantly produced, having two or more sequences, e.g., from unrelated genes arranged to make a new functional nucleic acid, e.g., a promoter from one source and a coding region from another source. Similarly, a heterologous protein will often refer to two or more subsequences that are not found in the same relationship to each other in nature (e.g., a fusion protein).

As used herein, the term “incubation product” refers to a solution that was held or incubated at an elevated temperature for a specific period of time.

As used herein, the term “inducer” is any compound that causes cells to produce larger amounts of enzymes or other substances than they would otherwise produce if the inducer was absent.

As used herein, the term “inducing feed”, refers to a solution fed to a microorganism that causes or induces the production of the desired protein product.

As used herein, the term “isolated” or “purified” refers to a nucleic acid or amino acid that is removed from at least one component with which it is naturally associated.

### IV. Induction of Gene Expression Using a High Concentration Sugar Mixture

This invention relates to methods for improved production of proteins from a cell culture. The invention also pertains to culture components and conditions that increase the amount of protein produced from genes under the control of certain inducible gene promoters. The improved methods can be used for the production of proteins encoded by naturally occurring cellulase genes as well as from various heterologous constructs. In particular, the invention relates to methods for improved production of proteins from a cell culture utilizing sucrose or other materials comprising sucrose as a carbon or sugar source.

### A. Induction of the Gene Expression

The filamentous fungus Trichoderma reesei is one of the most extensively studied cellulolytic organisms (reviewed e.g. by Nevalainen and Penttila, Mycota, 303-319, 1995). In industry, the cellulolytic enzymes of Trichoderma are used for many purposes including; production of fuel ethanol, paper, rayon, cellophane, detergents and fibers. Cellulase enzymes are also used to improve the nutritional value of animal feeds, and to facilitate the extraction of valuable components from plant cells (Mandels, Biochem. Soc. Trans., 414-16. 1985). Thus, these enzymes are of primary importance in the production of many useful products.

The production of cellulases in Trichoderma is dependent on the carbon source. Cellulose, lactose and the disaccharide sophorose, induce cellulase synthesis by Trichoderma reesei. Conversely, the presence of glucose results in tight repression of cellulase gene expression. Indeed, the expression level of the major cellobiohydrolase 1 (cbh1) is up-regulated several thousand folds on media containing inducing carbon sources such as cellulose or sophorose compared with glucose containing media (Ilmen et al., App. Environ. Microbio., 1298-1306, 1997).

Cellulase synthesis is subject to both cellulose induction and glucose repression. Thus, a critical factor influencing the yield of cellulase enzymes or heterologous proteins under the control of an inducible promoter is the maintenance of a proper balance between cellulose substrate and glucose concentration; it is critical for obtaining reasonable commercial yields of the regulated gene product. Although cellulose is an effective and inexpensive inducer, controlling the glucose concentration when Trichoderma is grown on solid cellulose can be problematic. At low concentrations of cellulose, glucose production may be too slow to meet the metabolic needs of active cell growth and function. On the other hand, cellulase synthesis can be halted by glucose repression when glucose generation is faster than consumption. Thus, expensive process control schemes are required to provide slow substrate addition and monitoring of glucose concentration (Ju and Afolabi, Biotechnol. Prog., 91-97, 1999). Moreover, the slow continuous delivery of substrate can be difficult to achieve as a result of the solid nature of the cellulosic materials.

Allen and Mortensen (Biotechnol. Bioeng., 2641-45, 1981) have shown that 200 IU/mL of purified β-glucosidase from Aspergillus phoenicis when incubated with a 50% glucose syrup produces a solution with the ability to induce cellulase production when used as a carbon source. Purification of the β-glucosidase is both time-consuming and expensive. In addition, these authors used more than 20× the β-glucosidase loading compared to that used in this current work.

Some of the problems associated with the use of cellulose as an inducing substrate can be overcome through the use of soluble substrates and inducers such as lactose or sophorose. Lactose has to be provided at high concentrations so as to function as an inducer and a carbon source. (See Seiboth, et. al., Mol. Genet. Genomics, 124-32, 2002.) Gentiobiose may also serve as an inducer. Sophorose is a more potent inducer than cellulose, but sophorose is expensive and difficult to manufacture. Published U.S. Pat. No. 7,713,725 disclosed when a whole cellulase preparation is added to a concentrated glucose solution, and the composition is incubated for at least two days at about 50° C. to about 75° C., a sugar mixture containing appreciable quantities of an inducer of cellulase gene expression is made, i.e., the inducing feed composition. The resulting mixture does not need any further purification and is competent to induce cellulase production as is.

### B. An Inducing Composition Comprising Sucrose Sources

Sucrose is made from two important sugar crops predominately: sugar cane (Saccharum spp.) and sugar beets (Beta vulgaris). Among the major sugar producing nations, including Brazil, India, European Union, China, Thailand, and United States (Food and Agriculture Organization of the United Nations. Retrieved on 2011 Nov. 18), Asia predominates in cane sugar production, with large contributions from India, China, Thailand, and other countries combining to account for 40% of global production of such cane sugars. South America comes in second place with 32% of global production, with Brazil being amongst or the largest sugar exporters in the world, exporting about 29 million tonnes in the year 2013, and the European Union (EU) being the world's second-largest sugar exporter.

Sucrose sources including sucrose molasses, an important residue of the sugar industry, are abundantly available in these parts of the world listed above. As such, it would be an advantage to have a convenient, soluble substrate composition using sucrose as one of the components, to also provide an inexpensive method of cellulase induction in filamentous fungi, such as, for example, Trichoderma reesei.

In a first aspect, the present disclosure provides a method of producing an inducing feed composition, said method comprising the steps of:
a) generating a first mixture by mixing sucrose and at least one inverting enzyme; and
b) incubating the first mixture at a suitable temperature for a sufficient period of time to produce an inverted mixture; and
c) generating a second mixture by mixing the inverted mixture produced in step b) with at least one reverting enzyme; and
d) incubating the second mixture at a suitable temperature for a sufficient period of time to produce an inducing feed composition.

The current invention provides a method of producing an inducing feed composition, using an inverted sugar mixture derived from sugar sources comprising sucrose.

An inverted sugar mixture is a mixture of equal parts of glucose and fructose resulting from the hydrolysis of sucrose. The sugar sources comprising sucrose may include sugarcane juice syrup, very high purity sucrose, sucrose molasses or any other sugar source comprising sucrose. The inverted sugar mixture may be obtained by inverting the sugar sources comprising sucrose using the enzyme invertase (EC 3.2.1.26), which catalyzes the hydrolysis of sucrose into glucose and fructose. The sucrose solution may be inverted at a temperature falling within the range of 30° C.-100° C., preferably within the range of 40° C.-90° C., such as within the range of 30° C.-90° C., or the range of 40° C.-80° C., or the range of 50° C.-80° C., or the range of 55° C.-75° C., for a period that is between 1 hour and 60 hours, preferably between 2 hours and 50 hours, such as, for example, for a period between 5 hours and 45 hours, or between 10 hours and 40 hours, or between 15 hours and 35 hours, and so on, while mixing with invertase.

An end-of-fermentation (EOF) broth (i.e., whole cellulase plus cells) may be added to an inverted sucrose solution. The presence of the cells or debris of cells would not affect the formation of sophorose. Accordingly there is no need to first recover the cellulases from the EOF broth before using the broth for producing a sophorose inducer. In fact, the enzyme mixture present in the fermentation broth at the end of the fermentation run can be used directly, without processing steps, as such even though the cells are still present in the broth.

In one embodiment, the invention provides a composition comprising an inverted sugar solution and a whole cellulase preparation that can be used as an inducing feed for the production of a protein of interest by a filamentous fungus. In one example, the protein of interest is a cellulolytic enzyme. In another example, the protein of interest is a protein heterologous to the filamentous fungal host. In a particular embodiment, the inducing feed induces cellulase enzyme production by Trichoderma reesei.

In one embodiment, a whole cellulase preparation from Trichoderma reesei is added to an inverted sugar solution to a final concentration of between 2 g and 20 g total protein/L. While the preferred range of final concentration may have been such, the final protein concentration of the mixture may range from as low as 0.5 g/L or as high as 50 g/L.

In one example the β-glucosidase activity in the inverted sugar solution is greater than 1.5 IU/mL. In one example the β-glucosidase activity in the inverted sugar solution is less than 200 IU/mL. In another example β-glucosidase activity of the inverted sugar solution is between 1.5 IU/mL and 200 IU/mL. In another example β-glucosidase activity of the inverted sugar solution is between 1.9 IU/mL and 200 IU/mL. In another example β-glucosidase activity of the inverted sugar solution is between 9.3 IU/mL and 200 IU/mL. In another example β-glucosidase activity of the inverted sugar solution is between 1.5 IU/mL and 180 IU/mL. In another example β-glucosidase activity of the inverted sugar solution is between 9.3 IU/mL and 180 IU/mL.

The mixture of whole cellulase or enriched cellulase and inverted sugar solution may be incubated at a temperature falling within the range of 30° C.-100° C., preferably in the range of 30° C.-90° C., for example in the range of 40° C.-90° C., or in the range of 50° C.-100° C., or in the range of 40° C.-80° C., or in the range of 50° C.-80° C., and so on.

The mixture of whole cellulase or enriched cellulase and inverted sugar solution may be incubated for a period between 2 hours and 7 days, for example, a period between 5 hours and 6 days, or between 10 hours and 5.5 days, or between 15 hours and 6 days, or between 20 hours and 7 days, and so on, with continuous or periodic mixing. In one example the incubation period is greater than about 2 days, for example, for 2 and half days, or 3 days or 4 days, or even 5 days or longer. In certain other examples, the incubation period is 2 days or shorter than 2 days, for example, for 1 and a half day, or 1 day, and so on. In further particular examples the incubation period is for about 3 days.

The sterilized final product solution from such an incubation can be harvested and used for fermentation feeding.

In certain embodiment the inducing feed is prepared by adding a whole cellulase preparation to the inverted sugar solution to a final concentration of 2 g total protein/L.

In a second aspect, the present disclosure provides a method for producing a protein of interest from a cell culture comprising: first, producing an inducing feed following the steps of (1) incubating a solution comprising about 50% to about 70% sucrose with at least one inverting enzyme, at a suitable temperature for a sufficient time period to generate a first inverted mixture; then (2) generating a second mixture by combining the first inverted mixture with at least one reverting enzyme, and incubating the mixture at a suitable temperature for a sufficient time period to form the inducing feed composition; and second, contacting a cell culture and the inducing feed composition at an amount sufficient or effective to induce expression of a protein of interest. The cell culture suitable comprises cells containing a nucleotide sequence encoding the protein of interest operatively linked to an inducible promoter.

### 1. Molecular Biology

In one embodiment this invention provides the expression of heterologous genes under control of the cellulase gene promoters of Trichoderma reesei. Therefore, this invention relies on routine techniques in the field of recombinant genetics. Basic texts disclosing the general methods of use in this invention include Sambrook et al., Molecular Cloning, A Laboratory Manual (2nd ed. 1989); Kriegler, Gene Transfer and Expression: A Laboratory Manual (1990); and Ausubel et al., eds., Current Protocols in Molecular Biology (1994)).

Heterologous genes comprising the cellulase promoter sequences of filamentous fungi are typically cloned into intermediate vectors before they are transformed into Trichoderma reesei cells for replication and/or expression. These intermediate vectors can be prokaryotic vectors, such as, e.g., plasmids, or shuttle vectors.

To obtain high level expression of a cloned gene, the heterologous gene is preferably positioned about the same distance from the promoter as is in the naturally occurring cellulase gene. As is known in the art, however, some variation in this distance can be accommodated without loss of promoter function.

Those skilled in the art are aware that a natural promoter can be modified by replacement, substitution, addition or elimination of one or more nucleotides without changing its function. The practice of the invention encompasses but is not constrained by such alterations to the promoter.

The expression vector/construct typically contains a transcription unit or expression cassette that contains all the additional elements required for the expression of the heterologous sequence. A typical expression cassette thus contains a promoter operably linked to the heterologous nucleic acid sequence and signals required for efficient polyadenylation of the transcript, ribosome binding sites, and translation termination. Additional elements of the cassette may include enhancers and, if genomic DNA is used as the structural gene, introns with functional splice donor and acceptor sites.

The practice of the invention is not constrained by the choice of promoter in the genetic construct. However, examples of suitable promoters include Trichoderma reesei cbh1, cbh2, eg1, egg, eg3, eg5, xln1, and/or xln2 promoters.

In addition to a promoter sequence, the expression cassette may also contain a transcription termination region downstream of the structural gene to provide for efficient termination. The termination region may be obtained from the same gene as the promoter sequence or may be obtained from different genes. Although any fungal terminator is likely to be functional in the present invention, preferred terminators include: the terminator from Trichoderma cbhI gene, the terminator from Aspergillus nidulans trpC gene (Yelton, M. et al. (1984) PNAS USA 81:1470-1474, Mullaney, E. J. et al. (1985) MGG 199:37-45), the Aspergillus awamori or Aspergillus niger glucoamylase genes (Nunberg, J. H. et al. (1984) Mol. Cell Biol. 4:2306, Boel, E. et al. (1984) EMBO J. 3:1581-1585), and/or the Mucor miehei carboxyl protease gene (EPO Publication No. 0 215 594).

The particular expression vector used to transport the genetic information into the cell is not particularly critical. Any of the conventional vectors used for expression in eukaryotic or prokaryotic cells may be used. Standard bacterial expression vectors include bacteriophages λ and M13, as well as plasmids such as pBR322 based plasmids, pSKF, pET23D, and fusion expression systems such as MBP, GST, and LacZ. Epitope tags can also be added to recombinant proteins to provide convenient methods of isolation, e.g., c-myc.

The elements that can be included in expression vectors may also be a replicon, a gene encoding antibiotic resistance to permit selection of bacteria that harbor recombinant plasmids, or unique restriction sites in nonessential regions of the plasmid to allow insertion of heterologous sequences. The particular antibiotic resistance gene chosen is not dispositive either, as any of the many resistance genes known in the art may be suitable. The prokaryotic sequences are preferably chosen such that they do not interfere with the replication or integration of the DNA in Trichoderma reesei.

The methods of transformation of the present invention may result in the stable integration of all or part of the transformation vector into the genome of the filamentous fungus. However, transformation resulting in the maintenance of a self-replicating extra-chromosomal transformation vector is also contemplated.

Many standard transfection methods can be used to produce Trichoderma reesei cell lines that express large quantities of the heterologus protein. Some of the published methods for the introduction of DNA constructs into cellulase-producing strains of Trichoderma include Lorito, Hayes, DiPietro and Harman, 1993, Curr. Genet. 24: 349-356; Goldman, VanMontagu and Herrera-Estrella, 1990, Curr. Genet. 17:169-174; Penttila, Nevalainen, Ratto, Salminen and Knowles, 1987, Gene 6: 155-164, for Aspergillus Yelton, Hamer and Timberlake, 1984, Proc. Natl. Acad. Sci. USA 81: 1470-1474, for Fusarium Bajar, Podila and Kolattukudy, 1991, Proc. Natl. Acad. Sci. USA 88: 8202-8212, for Streptomyces Hopwood et al., 1985, The John Innes Foundation, Norwich, UK and for Bacillus Brigidi, DeRossi, Bertarini, Riccardi and Matteuzzi, 1990, FEMS Microbiol. Lett. 55: 135-138).

Any of the known procedures for introducing foreign nucleotide sequences into host cells may be used. These include the use of calcium phosphate transfection, polybrene, protoplast fusion, electroporation, biolistics, liposomes, microinjection, plasma vectors, viral vectors and any of the other known methods for introducing cloned genomic DNA, cDNA, synthetic DNA or other foreign genetic material into a host cell (see, e.g., Sambrook et al., supra). Also of use is the Agrobacterium-mediated transfection method such as the one described in U.S. Pat. No. 6,255,115. It is only necessary that the particular genetic engineering procedure used be capable of successfully introducing at least one gene into the host cell capable of expressing the heterologous gene.

After the expression vector is introduced into the cells, the transfected cells are cultured under conditions favoring expression of genes under control of cellulase gene promoter sequences. Large batches of transformed cells can be cultured as described herein. Finally, product is recovered from the culture using standard techniques.

Thus, the invention herein provides for the expression and enhanced secretion of desired polypeptides whose expression is under control of cellulase gene promoter sequences including naturally occurring cellulase genes, fusion DNA sequences, and various heterologous constructs. The invention also provides processes for expressing and secreting high levels of such desired polypeptides.

### 2. Filamentous Fungi

Filamentous fungi include all filamentous forms of the subdivision Eumycota and Oomycota.

The filamentous fungi are characterized by vegetative mycelium having a cell wall composed of chitin, glucan, chitosan, mannan, and other complex polysaccharides, with vegetative growth by hyphal elongation and carbon catabolism that is obligately aerobic.

For purposes of the present invention, the filamentous fungal parent cell may be a cell of a species of, but not limited to, Trichoderma, e.g., Trichoderma longibrachiatum (reesei), Trichoderma viride, Trichoderma koningii, Trichoderma harzianum; Penicillium sp.; Humicola sp., including Humicola insolens; Chrysosporium sp., including C. lucknowense; Gliocladium sp.; Aspergillus sp.; Fusarium sp., Neurospora sp., Hypocrea sp., and Emericella sp.

As used herein, the term “Trichoderma” or “Trichoderma sp.” refers to any fungal strains which have previously been classified as Trichoderma or are currently classified as Trichoderma.

In certain embodiment, the filamentous fungal parent cell is an Aspergillus niger, Aspergillus oryzae, Aspergillus awamori, Aspergillus aculeatus, or Aspergillus nidulans cell.

In certain particular embodiment, the filamentous fungal parent cell is a Trichoderma reesei cell.

### 3. Protein Expression

Proteins of the present invention are produced by culturing cells transformed with an expression vector containing genes whose expression is under control of cellulase gene promoter sequences. The present invention is particularly useful for enhancing the intracellular and/or extracellular production of proteins. The protein may be homologous or heterologous. Proteins that may be produced by the instant invention include, but are not limited to, hormones, enzymes, growth factors, cytokines, antibodies and the like.

Enzymes include, but are not limited to, variously industrially useful enzymes such as protease, esterase, lipase, phenol oxidase, permease, amylase, glucoamylase, pullulanase, xylanase, cellulase, glucose isomerase, laccase, protein disulfide isomerase and the like.

Proteins of interest may include, for example, enzymes enzymes disclosed in PCT Application Publication Nos. WO03/027306, WO0200352118_A2, WO200352054_A2, WO200352057_A2, WO200352055_A2, WO200352056_A2, WO200416760_A2, WO9210581_A1, WO200448592_A2, WO200443980_A2, WO200528636_A2, WO200501065_A2, WO2005/001036, WO2005/093050, WO200593073_A1, WO200674005_A2, WO2009/149202, WO2011/038019, WO2010/141779, WO2011/063308, WO2012/125951, WO2012/125925, WO2012125937, WO/2011/153276, WO2014/093275, WO2014/070837, WO2014/070841, WO2014/070844, WO2014/093281, WO2014/093282, WO2014/093287, WO2014/093294, WO2015/084596, or WO2016/069541.

Hormones include, but are not limited to, follicle-stimulating hormone, luteinizing hormone, corticotropin-releasing factor, somatostatin, gonadotropin hormone, vasopressin, oxytocin, erythropoietin, insulin and the like.

Growth factors are proteins that bind to receptors on the cell surface, with the primary result of activating cellular proliferation and/or differentiation. Growth factors include, but are not limited to, platelet-derived growth factor, epidermal growth factor, nerve growth factor, fibroblast growth factors, insulin-like growth factors, transforming growth factors and the like.

Cytokines are a unique family of growth factors. Secreted primarily from leukocytes, cytokines stimulate both the humoral and cellular immune responses, as well as the activation of phagocytic cells. Cytokines include, but are not limited to, colony stimulating factors, the interleukins (IL-1 α and β, IL-2 through IL-13) and the interferons (α, β and γ).

Human Interleukin-3 (IL-3) is a 15 kDa protein containing 133 amino acid residues. IL-3 is a species specific colony stimulating factor which stimulates colony formation of megakaryocytes, neutrophils, and macrophages from bone marrow cultures.

Antibodies include, but are not limited to, immunoglobulins from any species from which it is desirable to produce large quantities. It is especially preferred that the antibodies are human antibodies. Immunoglobulins may be from any class, i.e., IgG, IgM, IgA, IgD or IgE.

Proteins of interest in the present invention may also be modified in a way to form chimeric molecules comprising a protein of interest fused to another, heterologous polypeptide or amino acid sequence. In one embodiment, such a chimeric molecule comprises a fusion of the protein of interest with a tag polypeptide which provides an epitope to which an anti-tag antibody can selectively bind. The epitope tag is generally placed at the amino- or carboxyl-terminus of the protein of interest.

Various tag polypeptides and their respective antibodies are well known in the art. Examples include poly-histidine (poly-his) or poly-histidine-glycine (poly-his-gly) tags; HIS6 and metal chelation tags, the flu HA tag polypeptide and its antibody 12CA5 (Field et al., Mol. Cell. Biol. 8:2159-2165 (1988)); the c-myc tag and the 8F9, 3C7, 6E10, G4, B7 and 9E10 antibodies thereto (Evan et al., Molecular and Cellular Biology 5:3610-3616 (1985)); and the Herpes Simplex virus glycoprotein D (gD) tag and its antibody (Paborsky et al., Protein Engineering 3(6):547-553 (1990)). Other tag polypeptides include the FLAG-peptide (Hopp et al., BioTechnology 6:1204-1210 (1988)); the KT3 epitope peptide (Martin et al., Science 255:192-194 (1992)); tubulin epitope peptide (Skinner et al., J. Biol. Chem. 266:15163-15166 (1991)); and the T7 gene 10 protein peptide tag (Lutz-Freyermuth et al., Proc. Natl. Acad. Sci. USA 87:6393-6397 (1990)).

In an alternative embodiment, the chimeric molecule may comprise a fusion of a protein of interest with an immunoglobulin or a particular region of an immunoglobulin. For a bivalent form of the chimeric molecule, such a fusion could be to the Fc region of an IgG molecule.

Conditions appropriate for expression of the genes of interest may include providing to the culture an inducing feed composition of the instant invention. Optimal conditions for the production of the proteins will vary with the choice of the host cell, and with the choice of protein to be expressed. Such conditions may be readily ascertained by one skilled in the art through routine experimentation or optimization.

The protein of interest can be purified or isolated after expression. The protein of interest may be isolated or purified in a variety of ways known to those skilled in the art depending on what other components are present in the sample. Standard purification methods include electrophoretic, molecular, immunological and chromatographic techniques, including ion exchange, hydrophobic, affinity, and reverse-phase HPLC chromatography, and chromatofocusing. For example, the protein of interest may be purified using a standard anti-protein of interest antibody column. Ultrafiltration and diafiltration techniques, in conjunction with protein concentration, are also useful. For general guidance in suitable purification techniques, see Scopes, Protein Purification (1982). The degree of purification necessary will vary depending on the use of the protein of interest. In some instances no purification will be necessary.

### 4. Fermentation

The invention relies on fermentation procedures for culturing fungi. Fermentation procedures for production of cellulase enzymes are known in the art. For example, cellulase enzymes can be produced either by solid or submerged culture, including batch, fed-batch and continuous-flow processes.

Culturing is accomplished in a growth medium comprising an aqueous mineral salts medium, organic growth factors, the carbon and energy source material, molecular oxygen, and, of course, a starting inoculum of one or more particular microorganism species to be employed.

In addition to the carbon and energy source, oxygen, assimilable nitrogen, and an inoculum of the microorganism, it is necessary to supply suitable amounts in proper proportions of mineral nutrients to assure proper microorganism growth, maximize the assimilation of the carbon and energy source by the cells in the microbial conversion process, and achieve maximum cellular yields with maximum cell density in the fermentation media.

The composition of the aqueous mineral medium can vary over a wide range, depending in part on the microorganism and substrate employed, as is known in the art. The mineral media should include, in addition to nitrogen, suitable amounts of phosphorus, magnesium, calcium, potassium, sulfur, and sodium, in suitable soluble assimilable ionic and combined forms, and also present preferably should be certain trace elements such as copper, manganese, molybdenum, zinc, iron, boron, and iodine, and others, again in suitable soluble assimilable form, all as known in the art.

The fermentation reaction is an aerobic process in which the molecular oxygen needed is supplied by a molecular oxygen-containing gas such as air, oxygen-enriched air, or even substantially pure molecular oxygen, provided to maintain the contents of the fermentation vessel with a suitable oxygen partial pressure effective in assisting the microorganism species to grow in a thriving fashion. In effect, by using an oxygenated hydrocarbon substrate, the oxygen requirement for growth of the microorganism is reduced. Nevertheless, molecular oxygen must be supplied for growth, since the assimilation of the substrate and corresponding growth of the microorganisms, is, in part, a combustion process.

Although the aeration rate can vary over a considerable range, aeration generally is conducted at a rate which is in the range of about 0.5 to 10, preferably about 0.5 to 7, volumes (at the pressure employed and at 25° C.) of oxygen-containing gas per liquid volume in the fermentor per minute. This amount is based on air of normal oxygen content being supplied to the reactor, and in terms of pure oxygen the respective ranges would be about 0.1 to 1.7, or preferably about 0.1 to 1.3, volumes (at the pressure employed and at 25° C.) of oxygen per liquid volume in the fermentor per minute.

The pressure employed for the microbial conversion process can range widely.

Pressures generally are within the range of about 0 to 50 psig, presently preferably about 0 to 30 psig, more preferably at least slightly over atmospheric pressure, as a balance of equipment and operating cost versus oxygen solubility achieved. Greater than atmospheric pressures are advantageous in that such pressures do tend to increase a dissolved oxygen concentration in the aqueous ferment, which in turn can help increase cellular growth rates. At the same time this is balanced by the fact that high atmospheric pressures do increase equipment and operating costs.

The fermentation temperature can vary somewhat, but for filamentous fungi such as Trichoderma reesei the temperature generally will be within the range of about 20° C. to 40° C., generally preferably in the range of about 25° C. to 34° C., depending on the strain of microorganism chosen.

The microorganisms also require a source of assimilable nitrogen. The source of assimilable nitrogen can be any nitrogen-containing compound or compounds capable of releasing nitrogen in a form suitable for metabolic utilization by the microorganism. While a variety of organic nitrogen source compounds, such as protein hydrolysates, can be employed, usually cheap nitrogen-containing compounds such as ammonia, ammonium hydroxide, urea, and various ammonium salts such as ammonium phosphate, ammonium sulfate, ammonium pyrophosphate, ammonium chloride, or various other ammonium compounds can be utilized. Ammonia gas itself is convenient for large scale operations, and can be employed by bubbling through the aqueous ferment (fermentation medium) in suitable amounts. At the same time, such ammonia can also be employed to assist in pH control.

The pH range in the aqueous microbial ferment (fermentation admixture) should be in the exemplary range of about 2.0 to 8.0. With filamentous fungi, the pH normally is within the range of about 2.5 to 8.0; with Trichoderma reesei, the pH normally is within the range of about 3.0 to 7.0. Preferences for pH range of microorganisms are dependent on the media employed to some extent, as well as the particular microorganism, and thus change somewhat with change in media as can be readily determined by those skilled in the art.

While the average retention time of the fermentation admixture in the fermentor can vary considerably, depending in part on the fermentation temperature and culture employed, generally it will be within the range of about 24 to 500 hours, preferably presently about 24 to 400 hours.

Preferably, the fermentation is conducted in such a manner that the carbon-containing substrate can be controlled as a limiting factor, thereby providing good conversion of the carbon-containing substrate to cells and avoiding contamination of the cells with a substantial amount of unconverted substrate. The latter is not a problem with water-soluble substrates, since any remaining traces are readily washed off. It may be a problem, however, in the case of non-water-soluble substrates, and require added product-treatment steps such as suitable washing steps.

As described above, the time to reach this level is not critical and may vary with the particular microorganism and fermentation process being conducted. However, it is well known in the art how to determine the carbon source concentration in the fermentation medium and whether or not the desired level of carbon source has been achieved.

Although the fermentation can be conducted as a batch or continuous operation, fed batch operation is much to be preferred for ease of control, production of uniform quantities of products, and most economical uses of all equipment.

If desired, part or all of the carbon and energy source material and/or part of the assimilable nitrogen source such as ammonia can be added to the aqueous mineral medium prior to feeding the aqueous mineral medium to the fermentor.

Each of the streams introduced into the reactor preferably is controlled at a predetermined rate, or in response to a need determinable by monitoring such as concentration of the carbon and energy substrate, pH, dissolved oxygen, oxygen or carbon dioxide in the off-gases from the fermentor, cell density measurable by dry cell weights, light transmittancy, or the like. The feed rates of the various materials can be varied so as to obtain as rapid a cell growth rate as possible, consistent with efficient utilization of the carbon and energy source, to obtain as high a yield of microorganism cells relative to substrate charge as possible.

In either a batch, or the preferred fed batch operation, all equipment, reactor, or fermentation means, vessel or container, piping, attendant circulating or cooling devices, and the like, are initially sterilized, usually by employing steam such as at about 121° C. for at least about 15 minutes. The sterilized reactor then is inoculated with a culture of the selected microorganism in the presence of all the required nutrients, including oxygen, and the carbon-containing substrate. The type of fermentor employed is not critical, though presently preferred is operation under 15L Biolafitte (Saint-Germain-en-Laye, France).

The collection and purification of the cellulase enzymes from the fermentation broth can also be done by procedures known per se in the art. The fermentation broth will generally contain cellular debris, including cells, various suspended solids and other biomass contaminants, as well as the desired cellulase enzyme product, which are preferably removed from the fermentation broth by means known in the art.

Suitable processes for such removal include conventional solid-liquid separation techniques such as, e.g., centrifugation, filtration, dialysis, microfiltration, rotary vacuum filtration, or other known processes, to produce a cell-free filtrate. It may be preferable to further concentrate the fermentation broth or the cell-free filtrate prior to crystallization using techniques such as ultrafiltration, evaporation or precipitation.

Precipitating the proteinaceous components of the supernatant or filtrate may be accomplished by means of a salt, e.g., ammonium sulfate, followed by purification by a variety of chromatographic procedures, e.g., ion exchange chromatography, affinity chromatography or similar art recognized procedures.

### EXAMPLES

The following examples are offered to illustrate, but not to limit the claimed invention.

The purpose of these experiments was to test sucrose sources as substrates for Trichoderma fermentations. A Trichoderma reesei strain's expressions of cellulase enzymes, using various sucrose feeds, was evaluated and compared. The sucrose feed included Sugarcane Juice Syrup (SJS), Very High Purity Sucrose (VHP) and Molasses (Mol).

### Example 1

### Glucose/Sophorose (G/S) Control Fermentation

The glucose/sophorose solution for induction of a control T. reesei protein production fermentation was prepared as previously described, in, for example, U.S. Pat. No. 7,713,725. The glucose/sophorose preparation was diluted to match the sugar solids content of the sucrose feeds. Solids content was 55%.

A typical industrial scale Trichoderma reesei fermentation process comprises two phases. First phase is the biomass growth phase, with little to no protein production; followed by the protein production phase, with little to no biomass growth. During the second phase, the rate of protein production is, in normal circumstances, linear. The protein production rate is the average rate from the start of the production phase to the end of the run. The yield calculation is the amount of protein produced during the protein production phase divided by the amount of sugars consumed during the same time period. Total protein in fermentation samples was measured using the Biuret method as modified by Weichselbaum and Gornall using Bovine Serum Albumin as a calibrator (Weichselbaum, T. Amer. J. Clin. Path. 1960, 16:40; Gornall, A. et al. J. Biol. Chem. 1949, 177:752).

The same calculations will be used for the sucrose fermentations described herein. The protein production phase ranges from 160 to 170 hours in each case. The results are presented as a percentage of the G/S control fermentation, in Table 1.

### Example 2

### Sugarcane Juice Syrup (SJS) Fermentation

Sugarcane juice syrup (sucrose) was inverted and reverted, and the resulting syrup was fed to T. reesei protein production fermentations. The results were compared to the control fermentation of the same strain, fed glucose/sophorose.

Three replicate fermentations were run using inverted and reverted Sugarcane Juice Syrup. Inversion was carried out at 75° C. and pH 2.0 for 4 hrs, followed by reversion for 48 hours at pH 4.0 and 60° C. with T. reesei beta-glucosidase (Bgl1) included at a concentration of 64,000 U/kg syrup. Alternatively inversion may be carried out employing a suitable invertase enzymes, for example, one derived from Aspergillus niger (UniPro Accession Number: QOZR36), Aspergillus fumigates, Aspergillus japonicas, Aspergillus nidulans, or other Aspergillus spp, or one derived from Fusarium oxyporum, or other like fungal species, as well as from various bacterial or even plant sources. The inversion reaction may be carried out at a suitable temperature such as in an acetate buffer, at pH 5-5.5, and a temperature of between 30 and 40° C., for an incubation (with mixing) period of between 1 hour and 48 hours.

The total solids content of the sugarcane juice syrup was 53%.

Inverted and reverted sugarcane juice syrup performed equivalent to glucose/sophorose for total protein production and yield of protein on sugars (Table 1).

### Example 3

### Very High Purity Sucrose (VHP) Fermentation

Very high purity (VHP) sucrose was inverted and reverted, and the resulting sugars fed to T. reesei protein production fermentations. The results were compared to the control fermentation of the same strain, fed glucose/sophorose.

Duplicate fermentations of T. reesei using the inverted and reverted VHP sucrose feed were performed. Inversion was carried out at 75° C. and pH 2.0 for 4 hrs, followed by reversion for 48 hours at pH 4.0 and 60° C. with T. reesei beta-glucosidase (Bgl1) included at a concentration of 64,000 U/kg syrup.

The total solids content of the VHP feed was 55%. A control fermentation was run in which the VHP inversion was conducted, but the reversion was omitted.

One of the VHP duplicate fermentations showed contamination in the final two time point samples. This did not appear to have a major effect on the performance. Yield was reduced by 3% relative to the non-contaminated run and rate was reduced by 7%. Omitting reversion after inversion resulted in much reduced protein production (Table 1).

The acidic conditions of reversion were insufficient to make required sophorose for optimal protein production.

### Example 4

### Sucrose Molasses (Mol) Fermentation

Sucrose molasses was inverted and reverted, and the resulting sugars fed to T. reesei protein production fermentations. The results were compared to the control fermentation of the same strain, fed glucose/sophorose.

Duplicate fermentations of T. reesei using inverted and reverted Sucrose Molasses feed were performed. Inversion was carried out at 75° C. and pH 2.0 for 4 hrs, followed by 48 hours at pH 4.0 and 60° C. with T. reesei beta-glucosidase (Bgl1) at 64,000 U/kg syrup. A third fermentation used a sucrose molasses inversion-only feed.

Without reversion, protein production was much reduced, again showing that the acidic conditions alone were insufficient for adequate sophorose production.

Protein production by molasses that was inverted and reverted was measured 27-38% above the G/S control. The same was observed in the yield of total protein on sugars (Table 1).

However, carbon and nitrogen balances showed more elements in products (cell mass, protein, and CO2) than in the substrates (sugars and NH₄OH), suggesting that there might have been a protein measurement error.

TABLE 1
Results of protein production and yield on sugars, from each
of the sucrose substrates tested, compared to the
glucose/sophorose (G/S) control.

| | | Protein | | Protein |
|---|---|---|---|---|
| | Protein | Production | Protein | Production |
| | Yield, | Rate, | Yield, | Rate, |
| | Relative to | Relative to | Average of | Average of |
| Fermentation | G/S Control | G/S Control | Replicates | Replicates |
| G/S control | 100% | 100% | — | — |
| SJS-1 | 94% | 95% | | |
| SJS-2 | 102% | 94% | | |
| SJS-3 | 105% | 99% | 100% | 96% |
| Mol | 60% | 46% | | |
| Inversion-only | | | | |
| Mol-1 | 130% | 124% | | |
| Mol-2 | 122% | 117% | 126% | 121% |
| VHP | 32% | 23% | | |
| Inversion-only | | | | |
| VHP-1 | 88% | 88% | | |
| VHP-2 | 85% | 82% | 87% | 85% |

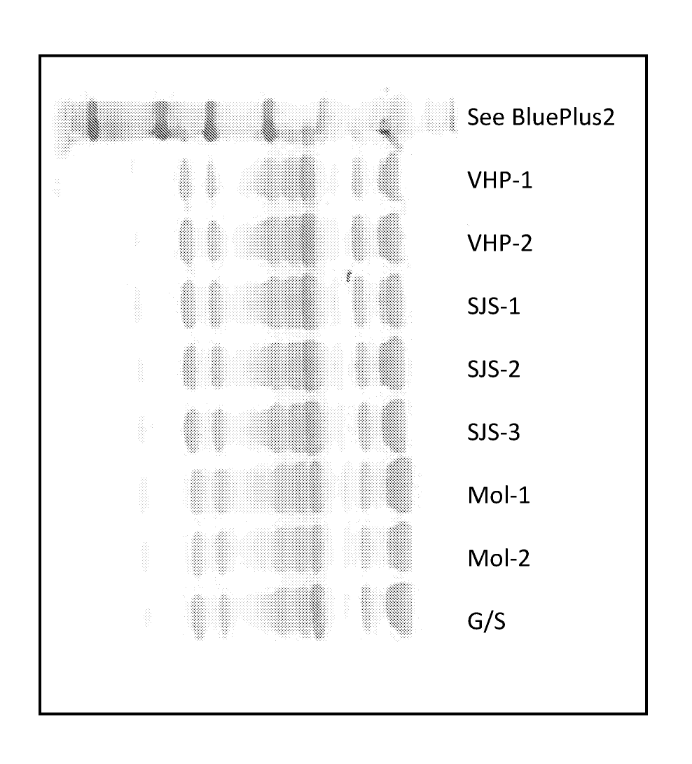

The protein products of each of the aforementioned fermentations were each diluted to 5 mg/mL based on the total protein measured by the modified Biuret method. They were prepared for SDS-PAGE analysis according to standard NuPAGE® protocols (Life Technologies) with LDS sample buffer, and run on a NuPAGE® 4-12% Bis-Tris gel with MOPS buffer. FIG. 1.

The protein products of each of the aforementioned fermentations were used in saccharification assays to demonstrate that effective enzyme compositions were produced by the various inverted and reverted sucrose feeds. The protein products of the fermentations fed sucrose that was only inverted were not tested for saccharification performance.

### Example 5

### Saccharification Performance

Saccharification of dilute ammonia pretreated corn stover was conducted as described herein, with the protein product of each of the described fermentations. The results were reported in percent glucan conversion, compared to the glucan content in the saccharification assay. The results shown in Table 2 are from the 14 mg protein/g glucan+xylan dose and 72 hrs incubation (other doses and time points were consistent).

### 5.1 Sugar Analysis by HPLC

Samples from saccharification assays were prepared by diluting 100 μL of saccharification hydrolysate into 900 μL of 0.01N H₂SO₄. These dilutions were then filtered using a Millipore MultiScreen_{HTS} GV MSGVN2250 vacuum filter plate. The filtrates were then transferred to a Nunc™ 96-well Polyproylene plate (Model #267245) and sealed using a Nunc™ 96-well Cap Mat (Model#276011).

This plate was then loaded into an Agilent G1377A autosampler for monomer sugar analysis using an Agilent 1200 HPLC.

Monomer sugars were determined by HPLC using a Bio-Rad Aminex™ HPX-87H (300×7.8 mm, 9 μm particle size, 8% crosslink) Ion-Exhange column with two Bio-Rad Micro-Guard Cation-H guard columns (30×4.6 mm). The solvent used was 0.01 N H₂SO₄, and the chromatography run was performed at an isocratic flow rate of 0.6 mL/min. The column temperature was maintained at 65° C. using the Agilent G1316A Thermosatted Column Compartment. Analyte detection was performed using an Agilent G1362A refractive index detector, at 50° C. internal temperature. The analysis time was about 18 minutes, with a sample injection volume was 20 μL.

A linear calibration curve was generated for glucose, xylose, and arabinose peak area versus concentration prior to sample analysis. A response factor for concentration to peak area was calculated from this calibration curve for glucose, xylose, and arabinose using Agilent ChemStation. The respective response factors for glucose, xylose and arabinose were then used to calculate the titers of each sugar in the prepared samples.

### 5.2 Biomass Saccharification Assay

Dilute ammonia pretreated corn stover, prepared in accordance with the method provided in, for example, published US Patent Application 20070031918, was used as the substrate in the saccharification assay. The assays were performed in glass scintillation vials. The final reaction mass was fixed at 5 g and 18% total solids.

The moisture content of the pretreated corn stover was measured using a Sartorius MA 45 infrared moisture analyzer (method detailed in National Renewable Energy Laboratory (NREL) Laboratory Analytical Procedure (LAP) described in NREL/TP-510-42621). Using this value, the appropriate amount of biomass substrate, e.g. the dilute ammonia pretreated corn stover, to give a final value of 18% solids was added to each vial. Reaction pH was adjusted using sulfuric acid to a pH of 5.30. Reaction pH was monitored daily and adjusted using either sulfuric acid or sodium hydroxide as necessary. The composition of structural carbohydrates in the reaction was 6.3% glucan and 3.9% xylan, measured using the NREL LAP as described in NREL/TP-510-42618 (see, http://www.nrel.gov/biomass/pdfs/42618.pdf).

After the pH was adjusted to pH 5.30, cellulase enzyme samples (described below) were added to the reaction vials. The enzyme dose was 10, 12, or 14 mg total protein per g of glucan and xylan. The appropriate volume of enzyme was added to each vial using an Eppendorf Research® plus variable volume pipette and VWR wide orifice tips. Sodium Azide, at a final concentration of 0.01% v/v, was added as an antimicrobial agent to all vials. To ensure that the final reaction solids were 18% solids and 5 g total reaction mass, the remaining volume after enzyme addition was balanced using ultrapure water.

Reaction vials were incubated at 50° C. and 200 rpm using an Innova 44 shaker-incubator for 72 hours. At the end of the 72 hour incubation, 1004, of each reaction was quenched in 9004, of 5 mM Sulfuric Acid, using an Eppendorf Research® plus variable volume pipette and VWR wide orifice tips. Samples were prepared for HPLC analysis as described above.

### 5.3 Arbocel Saccharification Assay

Arbocel® B600EU (manufactured by J. Rettenmaier & Söhne GmbH & Co KG, 73479 Rosenberg, Germany) saccharification assays were performed in glass scintillation vials. The final reaction mass was fixed at 5 g and 15% total solids. The moisture content of the Arbocel® substrate was measured using a Sartorius MA 45 infrared moisture analyzer (method detailed in NREL Laboratory Analytical Procedure (LAP) described in NREL/TP-510-42621, see, http://www.nrel.gov/biomass/pdfs/42618.pdf).

Using this value, the appropriate amount of biomass substrate to give a final value of 15% solids was added to each vial. Reaction pH was maintained using 50 mM Sodium Acetate Buffer (pH 5.0). The composition of structural carbohydrates in the reaction was 10.86% glucan and 3.1% xylan, measured using the NREL LAP described in NREL/TP-510-42618. After the pH was adjusted to pH 5.30, cellulase enzyme samples (described below) were added to the reaction vials. The enzyme dose was 10, 12, or 14 mg total protein per g of glucan and xylan. The appropriate volume of enzyme was added to each vial using an Eppendorf Research® plus variable volume pipette and VWR wide orifice tips. Sodium Azide, at a final concentration of 0.01% v/v, was added as an antimicrobial agent to all vials. To ensure that the final reaction solids was 15% solids and 5 g total reaction mass, the remaining volume after enzyme addition was balanced using ultrapure water.

Reaction vials were incubated at 50° C. and 200 rpm using an Innova 44 shaker-incubator for 17 hours. At the end of the 17 hour incubation, 100 μL of each reaction was quenched in 900 μL of 5 mM Sulfuric Acid, using an Eppendorf Research® plus variable volume pipette and VWR wide orifice tips. Samples were prepped for HPLC analysis as described above.

TABLE 2
Results of biomass saccharification by each of the fermentation
products, compared to the glucose/sophorose (G/S) control.

| Fermentation | Glucan Conversion (%) | Average of all replicates (%) |
|---|---|---|
| SJS-1 | 83.5 | 83.8 ± 1 |
| SJS-2 | 84.2 | |
| SJS-3 | 83.5 | |
| Mol-1 | 78.9 | 76.1 ± 3.3 |
| Mol-2 | 73.3 | |
| G/S Control | 82.8 | 82.8 ± 0.5 |
| VHP-1 | 72.2 | 74.3 ± 13.1 |
| VHP-2 | 76.1 | |

1. A method of producing an inducing feed composition, the method comprising the steps of:
a) generating a first mixture by mixing a sucrose-containing solution with at least one inverting enzyme;
b) incubating the first mixture at a first temperature for a first time period to produce an inverted mixture;
c) generating a second mixture by mixing the inverted mixture produced from b) and at least one reverting enzyme; and
d) incubating the second mixture at a second temperature for a second time period to produce the inducing feed composition.

2. The method of claim 1, wherein the sucrose-containing solution comprises Sugarcane Juice Syrup (SJS).

3. The method of claim 1, wherein the sucrose-containing solution comprises Very High Purity Sucrose (VHP).

4. The method of claim 1, wherein the sucrose-containing solution comprises Molasses (Mol).

5. The method of claim 1, wherein the inverting enzyme is an invertase.

6. The method of claim 1, wherein the first temperature is within the range of from about 30° C. to about 100° C., and the first time period is between 1 hour and 60 hours.

7. The method of claim 1, wherein the reverting enzyme is a whole cellulase composition comprising a beta-glucosidase.

8. The method of claim 7, wherein the reverting enzyme is a beta-glucosidase enriched cellulase composition.

9. The method of claim 1, wherein the second temperature is within the range of from about 30° C. to about 100° C., and the second time period is between 2 hours and 72 hours.

10. An inducing feed composition produced by applying the method of claim 1.

11. The inducing feed composition of claim 10, comprising a mixture of sugars.

12. The inducing feed composition of claim 11, comprising sophorose.

13. A method for producing a protein of interest from a cell culture comprising the steps of:
(a) producing an inducing feed composition, comprising the steps of:
(i) generating a first mixture by mixing a solution comprising from about 50% to about 70% sucrose and at least one inverting enzyme;
(ii) incubating the first mixture at a first temperature for a first time period to produce an inverted mixture; and
(iii) generating a second mixture by mixing the inverting mixture produced from (ii) and at least one reverting enzyme; and
(iv) incubating the second mixture at a second temperature for a second time period to produce the inducing feed composition;
(b) contacting the cell culture, wherein the cell culture comprises cells comprising a nucleotide sequence encoding a protein is interest operatively linked to an inducible promoter, with the inducing feed composition produced under step (a), in an amount effective to induce expression the protein of interest.

14. The method of claim 13, wherein the first temperature falls within the range of from about 30° C. to about 100° C., and the first time period is between 1 hour and 60 hours.

15. The method of claim 13, wherein the second temperature falls within the range of from about 30° C. to about 100° C., and the second time period is between 2 hours and 72 hours.

16. The method of claim 13, wherein the protein of interest is a protein that is endogenous to the cell culture.

17. The method of claim 13, wherein the protein of interest is a protein that is heterologous to the cell culture.

18. The method of claim 13, wherein the protein of interest is selected from the group consisting of hemicellulases, peroxidases, proteases, cellulases, xylanases, lipases, phospholipases, esterases, cutinases, pectinases, keratinases, reductases, oxidases, phenol oxidases, lipoxygenases, ligninases, pullulanases, tannases, pentosanases, mannanases, beta-glucanases, arabinosidases, hyaluronidase, chondroitinase, laccase, amylases, glucoamylases, and mixtures thereof.

19. The method of claim 13, wherein the inducible promoter is a sophorose-inducible promoter.

20. The method of claim 13, wherein the promoter is a cellulase gene promoter.

21. The method of claim 20, wherein the promoter is a cbh 1 promoter from Trichoderma reesei.

22. The method of claim 15, wherein the cell of the cell culture is a filamentous fungal cell.

23. The method of claim 22, wherein the fungus is a Trichoderma spp.

24. The method of claim 22, wherein the fungus is a Penicillium spp.

25. The method of claim 22, wherein the fungus is an Aspergillus spp.
